(19) United States
(12) Patent Application Publication
(10) Pub. No.: US 2023/0277706 A1
(43) Pub. Date: Sep. 7, 2023

(54) DECONTAMINATION OF A SURFACE OF A MATERIAL

(71) Applicant: The Boeing Company, Chicago, IL (US)

(72) Inventors: Alexander G. Dunlap, Huntsville, AL (US); Jeffrey D. Poskin, Black Diamond, WA (US); Sabyasachi Basu, Kingston, WA (US)

(21) Appl. No.: 17/653,707

(22) Filed: Mar. 7, 2022

(51) Int. Cl. A61L 2/24; A61L 2/22; A61L 2/28

(52) U.S. Cl. CPC A61L 2/24; A61L 2/22; A61L 2/28; A61L 2202/14; A61L 2202/15

(57) ABSTRACT

A method of decontaminating a surface of a material includes obtaining training data including thermographic images of a test surface on which a liquid with a decontamination agent is deposited, the thermographic images including intensity values that describe radiation emitted across the test surface over time. The method includes accessing a parametric model of the radiation emitted as a function of time, and applying a spatial regularization to the parametric model in which parameters of the parametric model are made functions of location across the test surface of the material. The parametric model with the spatial regularization is fit to the training data to produce a fitted parametric model. And the fitted parametric model is deployed to predict the radiation emitted across a working surface on which the liquid is deposited, and decontamination of the working surface is determinable from the radiation emitted as predicted.

### TECHNOLOGICAL FIELD

The present disclosure relates generally to decontamination and, in particular, to decontamination of a surface of a material such as an interior surface of an aircraft.

### BACKGROUND

Recent pandemics of Severe Acute Respiratory Syndrome (SARS) and coronavirus disease 2019 (COVID-19) have revealed vulnerabilities regarding global disease transmission, and its effect on the global economy. One industry that has been affected is the transportation industry that includes the airline industry. The Centers for Disease Control and Prevention (CDC) and regulators in the transportation industry including the Federal Aviation Administration (FAA) have recommended precautions to avoid exposure to disease, and these precautions include decontamination of the interior surfaces of aircraft.

Various techniques exist for decontamination of a surface of a material such as an interior surface of an aircraft. One technique involves use of a liquid that is deposited on the surface. The liquid includes a decontamination agent that acts to remove contaminants from the surface, such as by chemical reaction or disinfection. One measure of the effectiveness of the decontamination agent in removing contaminants is the amount of time the surface remains wet from the liquid deposited on the surface. This may also be referenced by the drying time of the surface on which the liquid is deposited. The drying time, however, depends on a number of factors and is not easily determined beforehand.

It would therefore be desirable to have a system and method that takes into account at least some of the issues discussed above, as well as other possible issues.

### BRIEF SUMMARY

Example implementations of the present disclosure are directed to decontamination and, in particular, to decontamination of a surface of a material such as an interior surface of an aircraft. In particular, example implementations are directed to producing a fitted, parametric model that may be deployed to predict radiation emitted across a surface of a material over time, which may indicate temperature across the test surface over time. In cases in which a liquid is deposited on the surface, the radiation as predicted may be used to determine a drying time for the surface by evaporation of the liquid. And when the liquid includes a decontamination agent, example implementations may determine that the surface is decontaminated when the drying time is at least a specified decontamination time.

The present disclosure thus includes, without limitation, the following example implementations.

Some example implementations provide an apparatus for decontaminating a surface of a material, the apparatus comprising: a memory configured to store computer-readable program code; and processing circuitry configured to access the memory, and execute the computer-readable program code to cause the apparatus to at least: obtain training data including a time-lapse of thermographic images of a test surface of the material on which a liquid that includes a decontamination agent is deposited, the thermographic images having a dot matrix data structure with a matrix of intensity values that describe radiation emitted and thereby indicate temperature across the test surface over time; access a parametric model of the radiation emitted as a function of time, the parametric model including a fixed set of parameters with values that are unknown; apply a spatial regularization to the parametric model in which the fixed set of parameters are made functions of location across the test surface of the material; fit the parametric model with the spatial regularization to the training data to estimate the values of the fixed set of parameters, and thereby produce a fitted parametric model, and deploy the fitted parametric model to predict the radiation emitted across a working surface of the material on which the liquid is deposited, the radiation emitted is predicted across the working surface over time, and decontamination of the working surface is determinable from the radiation emitted as predicted.

Some example implementations provide a method of decontaminating a surface of a material, the method comprising: obtaining training data including a time-lapse of thermographic images of a test surface of the material on which a liquid that includes a decontamination agent is deposited, the thermographic images having a dot matrix data structure with a matrix of intensity values that describe radiation emitted and thereby indicate temperature across the test surface over time; accessing a parametric model of the radiation emitted as a function of time, the parametric model including a fixed set of parameters with values that are unknown; applying a spatial regularization to the parametric model in which the fixed set of parameters are made functions of location across the test surface of the material; fitting the parametric model with the spatial regularization to the training data to estimate the values of the fixed set of parameters, and thereby produce a fitted parametric model; and deploying the fitted parametric model to predict the radiation emitted across a working surface of the material on which the liquid is deposited, the radiation emitted is predicted across the working surface over time, and decontamination of the working surface is determinable from the radiation emitted as predicted.

Some example implementations provide a computer-readable storage medium for decontaminating a surface of a material, the computer-readable storage medium being non-transitory and having computer-readable program code stored therein that, in response to execution by processing circuitry, causes an apparatus to at least: obtain training data including a time-lapse of thermographic images of a test surface of the material on which a liquid that includes a decontamination agent is deposited, the thermographic images having a dot matrix data structure with a matrix of intensity values that describe radiation emitted and thereby indicate temperature across the test surface over time; access a parametric model of the radiation emitted as a function of time, the parametric model including a fixed set of parameters with values that are unknown; apply a spatial regularization to the parametric model in which the fixed set of parameters are made functions of location across the test surface of the material; fit the parametric model with the spatial regularization to the training data to estimate the values of the fixed set of parameters, and thereby produce a fitted parametric model; and deploy the fitted parametric model to predict the radiation emitted across a working surface of the material on which the liquid is deposited, the radiation emitted is predicted across the working surface over time, and decontamination of the working surface is determinable from the radiation emitted as predicted.

These and other features, aspects, and advantages of the present disclosure will be apparent from a reading of the following detailed description together with the accompanying figures, which are briefly described below. The present disclosure includes any combination of two, three, four or more features or elements set forth in this disclosure, regardless of whether such features or elements are expressly combined or otherwise recited in a specific example implementation described herein. This disclosure is intended to be read holistically such that any separable features or elements of the disclosure, in any of its aspects and example implementations, should be viewed as combinable unless the context of the disclosure clearly dictates otherwise.

It will therefore be appreciated that this Brief Summary is provided merely for purposes of summarizing some example implementations so as to provide a basic understanding of some aspects of the disclosure. Accordingly, it will be appreciated that the above described example implementations are merely examples and should not be construed to narrow the scope or spirit of the disclosure in any way. Other example implementations, aspects and advantages will become apparent from the following detailed description taken in conjunction with the accompanying figures which illustrate, by way of example, the principles of some described example implementations.

### BRIEF DESCRIPTION OF THE FIGURE(S)

Having thus described example implementations of the disclosure in general terms, reference will now be made to the accompanying figures, which are not necessarily drawn to scale, and wherein:

FIG. 1 illustrates one type of vehicle, namely, an aircraft that may benefit from example implementations of the present disclosure;

FIG. 2 illustrates an aircraft manufacturing and service method, according to some example implementations;

FIG. 3 illustrates graphs of intensity value profiles for two different materials, namely Tedlar® and wool, according to some example implementations;

FIG. 4 is a graph that illustrates an example of intensity values at a position in a matrix of intensity values over time, according to some example implementations;

FIG. 5 is a graph that illustrates how dry-time may be determined based on intensity value using a parametric model, according to some example implementations;

FIGS. 6A, 6B, 6C, 6D, 6E and 6F are flowcharts illustrating various steps in a method of decontaminating a surface of a material, according to example implementations; and

FIG. 7 illustrates an apparatus according to some example implementations.

### DETAILED DESCRIPTION

Some implementations of the present disclosure will now be described more fully hereinafter with reference to the accompanying figures, in which some, but not all implementations of the disclosure are shown. Indeed, various implementations of the disclosure may be embodied in many different forms and should not be construed as limited to the implementations set forth herein; rather, these example implementations are provided so that this disclosure will be thorough and complete, and will fully convey the scope of the disclosure to those skilled in the art. Like reference numerals refer to like elements throughout.

Unless specified otherwise or clear from context, references to first, second or the like should not be construed to imply a particular order. A feature described as being above another feature (unless specified otherwise or clear from context) may instead be below, and vice versa; and similarly, features described as being to the left of another feature else may instead be to the right, and vice versa. Also, while reference may be made herein to quantitative measures, values, geometric relationships or the like, unless otherwise stated, any one or more if not all of these may be absolute or approximate to account for acceptable variations that may occur, such as those due to engineering tolerances or the like.

As used herein, unless specified otherwise or clear from context, the “or” of a set of operands is the “inclusive or” and thereby true if and only if one or more of the operands is true, as opposed to the “exclusive or” which is false when all of the operands are true. Thus, for example, “[A] or [B]” is true if [A] is true, or if [B] is true, or if both [A] and [B] are true. Further, the articles “a” and “an” mean “one or more,” unless specified otherwise or clear from context to be directed to a singular form. Furthermore, it should be understood that unless otherwise specified, the terms “data,” “content,” “digital content,” “information,” and similar terms may be at times used interchangeably.

Example implementations of the present disclosure relate generally to vehicular engineering and, in particular, to one or more of the design, construction, operation or use of vehicles. As used herein, a vehicle is a machine designed as an instrument of conveyance by land, water or air. A vehicle designed and configurable to fly may at times be referred to as an aerial vehicle, an aircraft or the like. Other examples of suitable vehicles include any of a number of different types of ground vehicles (e.g., motor vehicles, railed vehicles), watercraft, amphibious vehicles, spacecraft and the like.

A vehicle generally includes a basic structure, and a propulsion system coupled to the basic structure. The basic structure is the main supporting structure of the vehicle to which other components are attached. The basic structure is the load-bearing framework of the vehicle that structurally supports the vehicle in its construction and function. In various contexts, the basic structure may be referred to as a chassis, an airframe or the like.

The propulsion system includes one or more engines or motors configured to power one or more propulsors to generate propulsive forces that cause the vehicle to move. A propulsor is any of a number of different means of converting power into a propulsive force. Examples of suitable propulsors include rotors, propellers, wheels and the like. In some examples, the propulsion system includes a drivetrain configured to deliver power from the engines/motors to the propulsors. The engines/motors and drivetrain may in some contexts be referred to as the powertrain of the vehicle.

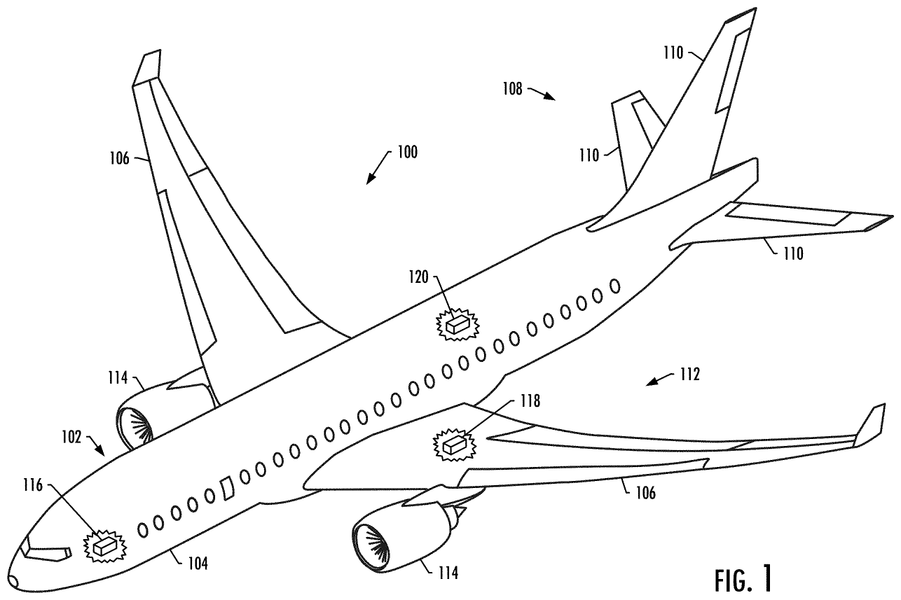

FIG. 1 illustrates one type of vehicle, namely, an aircraft 100 that may benefit from example implementations of the present disclosure. As shown, the aircraft includes a basic structure with an airframe 102 including a fuselage 104. The airframe also includes wings 106 that extend from opposing sides of the fuselage, an empennage or tail assembly 108 at a rear end of the fuselage, and the tail assembly includes stabilizers 110. The aircraft also includes a plurality of high-level systems 112 such as a propulsion system. In the particular example shown in FIG. 1, the propulsion system includes two wing-mounted engines 114 configured to power propulsors to generate propulsive forces that cause the aircraft to move. In other implementations, the propulsion system can include other arrangements, for example, engines carried by other portions of the aircraft including the fuselage and/or the tail. As also shown, the high-level systems may also include an electrical system 116, hydraulic system 118 and/or environmental system 120. Any number of other systems may be included.

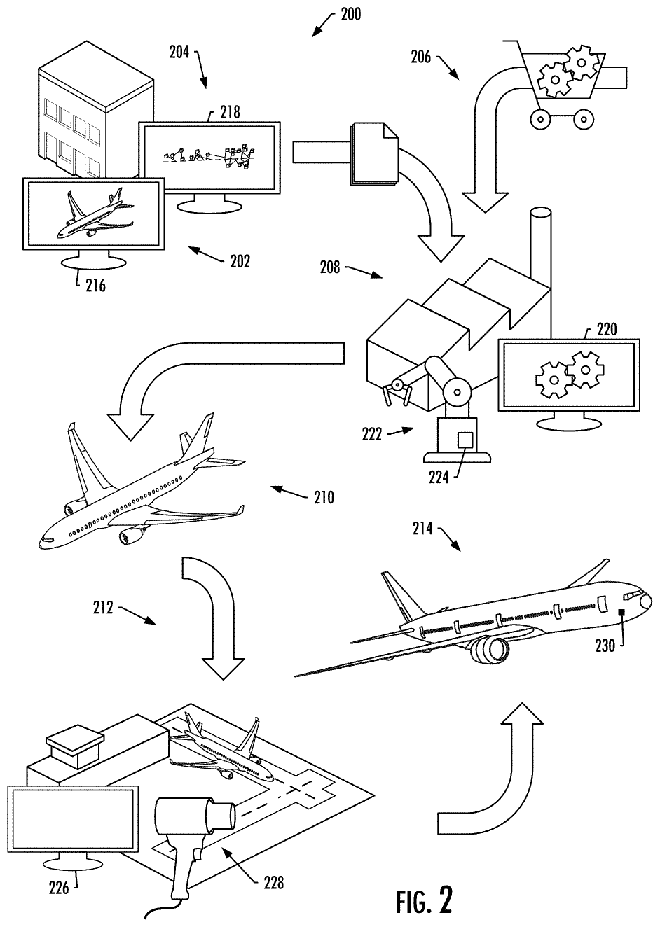

As explained above, example implementations of the present disclosure relate generally to vehicular engineering and, in particular, to one or more of the design, construction, operation or use of vehicles such as aircraft 100. Thus, referring now to FIG. 2, example implementations may be used in the context of an aircraft manufacturing and service method 200. During pre-production, the example method may include specification and design 202 of the aircraft, manufacturing sequence and processing planning 204 and material procurement 206. During production, component and subassembly manufacturing 208 and system integration 210 of the aircraft takes place. Thereafter, the aircraft may go through certification and delivery 212 in order to be placed in service 214. While in service by an operator, the aircraft may be scheduled for maintenance and service (which may also include modification, reconfiguration, refurbishment or the like).

Each of the processes of the example method 200 may be performed or carried out by a system integrator, third party and/or operator (e.g., customer). For the purposes of this description, a system integrator may include for example any number of aircraft manufacturers and major-system subcontractors; a third party may include for example any number of vendors, subcontractors and suppliers; and an operator may include for example an airline, leasing company, military entity, service organization or the like.

As will also be appreciated, computers are often used throughout the method 200; and in this regard, a “computer” is generally a machine that is programmable or programmed to perform functions or operations. The method as shown makes use of a number of example computers. These computers include computers 216, 218 used for the specification and design 202 of the aircraft, and the manufacturing sequence and processing planning 204. The method may also make use of computers 220 during component and subassembly manufacturing 208, which may also make use of computer numerical control (CNC) machines 222 or other robotics that are controlled by computers 224. Even further, computers 226 may be used while the aircraft is in service 214, as well as during maintenance and service, any or all of which may also make use of a sprayer such as an electrostatic sprayer 228 to decontaminate interior surfaces of the aircraft. And as suggested in FIG. 1, the aircraft may itself include one or more computers 230 as part of or separate from its electrical system 116.

A number of the computers 216, 218, 220, 224, 226 and 230 used in the method 200 may be co-located or directly coupled to one another, or in some examples, various ones of the computers may communicate with one another across one or more computer networks. Further, although shown as part of the method, it should be understood that any one or more of the computers may function or operate separate from the method, without regard to any of the other computers. It should also be understood that the method may include one or more additional or alternative computers than those shown in FIG. 2.

Example implementations of the present disclosure may be implemented throughout the aircraft manufacturing and service method 200, including during pre-production, production or in-service. In this regard, some example implementations provide a computer such as computer 226 for decontaminating a surface of a material such as an interior surface of the aircraft 100; and in particular, for use to determine decontamination of the surface of the material. Although described with respect to computer 226, it should be understood that example implementations may be equally implemented by any of the computers 216, 218, 220, 224, 226 or 230 used in the method 200.

According to example implementations, the computer 226 for use to facilitate decontamination of a surface of a material. The computer is configured to obtain training data including a time-lapse of thermographic images of a test surface of the material on which a liquid that includes a decontamination agent (at times referred to as a “decontaminant”) is deposited. In some examples, the liquid with the decontamination agent is deposited on the test surface in an environment and using an electrostatic sprayer 228. The thermographic images may be in color or grayscale; and in some examples, the thermographic images may be in color, and converted to grayscale. The thermographic images have a dot matrix data structure with a matrix of intensity values that describe radiation emitted and thereby indicate temperature across the test surface over time. These intensity values may correspond to pixels of the thermographic image; and in grayscale, the intensity values may take the form of 8-bit unsigned integers each taking on a value from 0-255.

Intensity value as a function of time exhibit a common pattern after being sprayed. Typical intensity value profiles for two different materials, namely Tedlar® and wool, are shown in FIG. 3. At the time of spray there is a rapid drop in intensity value, followed by a more gradual drop and rise in intensity during what may be referred to as cooling and warming phases. These phases are clearly shown in the Tedlar® example of FIG. 3. The cooling phase is presumably caused when the thin film of liquid deposited by the electrostatic sprayer is evaporating from the surface of the target object. During the warming phase, the intensity value quickly ramps up and eventually levels off as that portion of the target object reaches thermal equilibrium. The duration of these phases can vary depending on the thickness of the deposited film, the material properties of the material, and environmental conditions such as temperature and humidity. The profile for wool as shown FIG. 3, for example, has almost no cooling phase.

Given this understanding of pixel intensity as a function of time, the computer 226 may model the radiation emitted as a function of time. In this regard, the computer is configured to access a parametric model of the radiation emitted as a function of time, the parametric model including a fixed set of parameters with values that are unknown. The computer is configured to apply a spatial regularization to the parametric model in which the fixed set of parameters are made functions of location across the test surface of the material. The computer is configured to fit the parametric model with the spatial regularization to the training data to estimate the values of the fixed set of parameters, and thereby produce a fitted parametric model. And the computer is configured to deploy the fitted parametric model to predict the radiation emitted across a working surface of the material on which the liquid is deposited such as using an electrostatic sprayer 228, the radiation emitted is predicted across the working surface over time. Decontamination of the working surface, then, is determinable from the radiation emitted as predicted.

In some examples, the computer 226 is further configured to predict a drying time for the working surface of the material by evaporation of the liquid, from the radiation emitted as predicted across the working surface over time. And in some of these examples, the computer is further configured to determine that the working surface of the material is decontaminated when the drying time is at least a specified decontamination time.

In some examples, the training data including the time-lapse of thermographic images is obtained from an experiment designed to test an effect of a number of experimental factors on the radiation emitted across the test surface over time. In some of these examples, then, the fitted parametric model is deployed to predict the radiation emitted under particular levels of the number of experimental factors. Examples of suitable experimental factors include multiple ones of the material, a profile of the test surface of the material, a temperature of the environment, a humidity of the environment, the electrostatic sprayer 228, the liquid, or an orientation of the electrostatic sprayer with respect to the test surface when the liquid with the decontamination agent is deposited.

In some examples, the experiment is also designed to test the effect of the number of experimental factors on coverage of the liquid across the test surface. In some of these examples, the computer 226 is further configured to carry out the experiment to determine the particular levels of the number of experimental factors that optimize the coverage of the liquid.

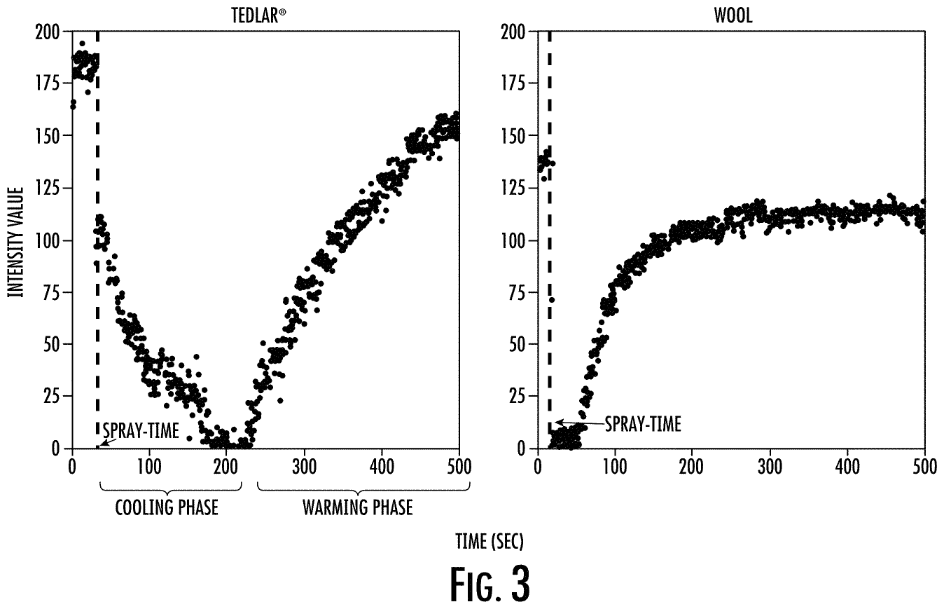

In some further examples, the thermographic images include a thermographic image captured before the liquid is deposited on the test surface, and a later thermographic image captured after the liquid is deposited on the test surface. The computer 226, then, is configured to determine the coverage of the liquid across the test surface from the thermographic image and the later thermographic image. More particularly, for example, the computer is configured to access the matrix of intensity values of the thermographic image and the later thermographic image, and compare the intensity values at respective positions in the matrix of intensity values to determine those of the intensity values that have a statistically significant difference between the thermographic image and the later thermographic image, which indicates coverage of the liquid across the test surface. FIG. 4 illustrates an example of intensity values at a position in the matrix of intensity values over time, which may be used to determine coverage of the liquid.

In a more specific example, p-values may be calculated for intensity values at a position between the thermographic image and the later thermographic image, using a two-sample t-test and corrected for multiple tests to have a false discovery rate of 0.05 using Benjamini-Hochberg. And coverage of the liquid may be reported as a percentage of the corresponding intensity values that had a significant change.

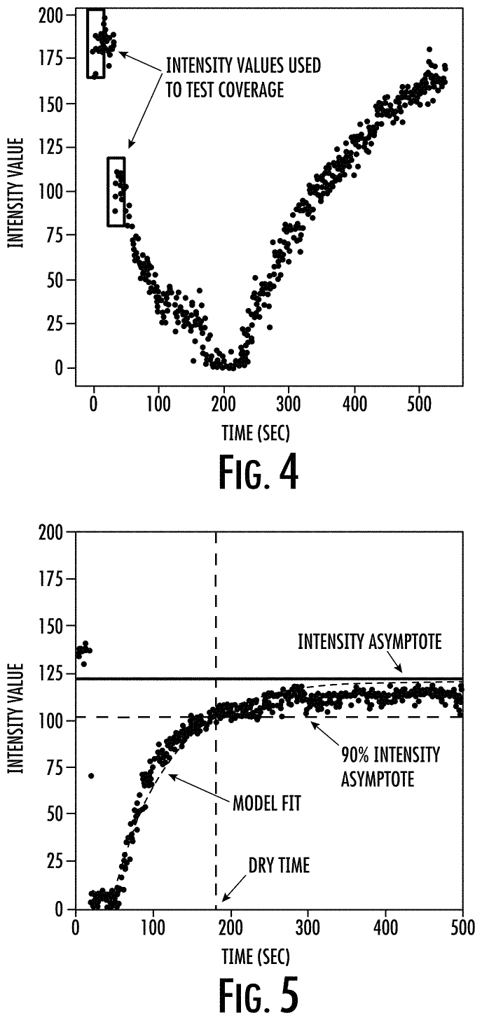

To further illustrate various example implementations of the present disclosure in which an electrostatic sprayer 228 is used to spray or otherwise deposit a liquid with a decontamination agent on the surface of a material, FIG. 5 is a graph that illustrates how dry-time may be determined based on intensity value using a parametric model. In some examples, the parameteric model of the radiation emitted u as a function of time t may be given by the map u(t; a₁, a₂, a₃, b₁, b₂, b₃, γ): [symbol] ⁸→ [symbol] having the form

$$u ( t ; {a}_{1} , {a}_{2} , {a}_{3} , {b}_{1} , {b}_{2} , {b}_{3} , γ ) = \{ \begin{matrix}{a}_{1} + {a}_{2} t + {a}_{3} \sqrt{t} , t \leq γ \\ {b}_{1} - exp ( {b}_{2} t + {b}_{3} ) , t > γ\end{matrix}$$

where a₁, a₂, a₃, b₁, b₂, b₃, γ∈ [symbol] are model parameters. In particular, a₁, a₂ and a₃ are model parameters for the evaporative cooling phase, b₁, b₂ and b₃ are model parameters for the warming phase, and γ is a model parameter that represents the breakpoint time where the model switches from cooling to warming.

In some examples, the parameters a₂, a₃, and b₂ are constrained to be less than zero. The parametric model may also make a number of simplifying assumptions. The evaporative cooling phase may assume the target object is a sufficiently thick semi-infinite body, and the warming phase may assume conductive heat transfer with no spatial variation and an energy rate density proportional to the difference between the target object and the temperature of the environment.

To get a further functional form of the parametric model, the breakpoint may be estimated by adding the constraint

a₁+a₂γ+a₃\sqrt{γ}=b₁−exp(b₂γ+b₃)

a₁+a₂γ+a₃\sqrt{γ}+exp(b₂γ+b₃)=b₁

and implementing a piecewise function using a maximum so that for a position p in the matrix of intensity values for a surface S, the intensity value uₚ may be given by

$${u}_{p} ( t ; {a}_{1} , {a}_{2} , {a}_{3} , {b}_{2} , {b}_{3} , γ ) = max \{ \begin{matrix}{a}_{1} + {a}_{2} + {a}_{3} \sqrt{t} , \\ {a}_{1} + {a}_{2} γ + {a}_{3} \sqrt{γ} + exp ( {b}_{2} γ + {b}_{3} ) - exp ( {b}_{2} t + {b}_{3} )\end{matrix}$$

In this situation each model parameter depends only on the intensity measurements at position p, and not on the location of p on S. That is, each position p has a set of model parameters a₁^{p}, a₂^{p}, a₃^{p}, b₂^{p}, b₃^{p}, γ^{p} that predict the intensity value at the position over time. However, physical knowledge of the problem suggests that surface should exhibit similar drying behavior in local regions. This knowledge may be incorporated into the above model by approximating model parameters, a₁, . . . , γ, as continuous functions defined across the surface S. For example, the function a₁: S→ [symbol] may provide the model parameter a₁(p)=a₁^{p} for position p on the surface.

In some examples, tensor-product splines may be used to approximate these model parameter functions. These approximations may be obtained by solving a least squares fit to intensity value data that includes an additional spatial regularization term. The resulting optimization problem, then, may be as presented below.

Let pᵢⱼₜ represent the intensity value for position (i,j) at time t in a dataset collected by a thermographic camera. Let

$$u ( i , j , t ) = max \{ \begin{matrix}{a}_{1} ( i , j ) + {a}_{2} ( i , j ) + {a}_{3} ( i , j ) \sqrt{t} , \\ \begin{matrix}{a}_{1} ( i , j ) + {a}_{2} ( i , j ) γ + {a}_{3} ( i , j ) \sqrt{γ} + \\ exp ( {b}_{2} ( i , j ) γ + {b}_{3} ( i , j ) - exp ( {b}_{2} ( i , j ) t + {b}_{3} ( i , j )\end{matrix}\end{matrix}$$

be the model's predicted intensity value of position (i,j) at time t. Let

$$E ( {a}_{1} ) = \int \int {( \frac{{\partial }^{2} {a}_{1}}{\partial {x}^{2}} )}^{2} + 2 {( \frac{{\partial }^{2} {a}_{1}}{\partial x \partial y} )}^{2} + {( \frac{{\partial }^{2} {a}_{1}}{\partial {y}^{2}} )}^{2} dxdy$$

be a thin plate spline energy functional that measures variation in the function a₁. Let [symbol] ={a₁, a₂, a₃, b₂, b₃, γ} denote the set of model parameter functions determined by a collection of coefficients c over some parameterization C of a fixed function space of tensor product splines.

A set of approximations to intensity value data, then, may be determined from the following:

$${min}_{c ∈ C} {\sum }_{i , j , t} {( {p}_{ijt} - u ( i , j , t ) )}^{2} + μ ℰ ( c )$$

where ε(c)=E(a₁)+E(a₂) . . . +E(γ).

An example model fit is shown in FIG. 5. Also shown is the value for the asymptote of the warming phase, which may be used to determine the dry-time by finding the point where the model curve crosses a threshold (e.g., 90%) of the warming asymptote. Setting the threshold to a particular value such as 90% of the warming asymptote is a heuristic, which may be rationalized since it is where the pixel intensity is “close enough” to thermal equilibrium and well past the point governed by evaporative cooling. The drying time, then, may be reported as the 90th percentile of the dry-times for all positions in the matrix of intensity values of the thermographic images.

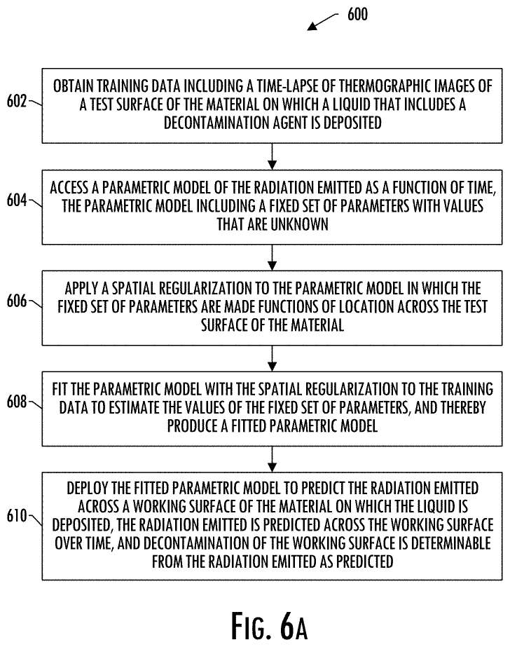

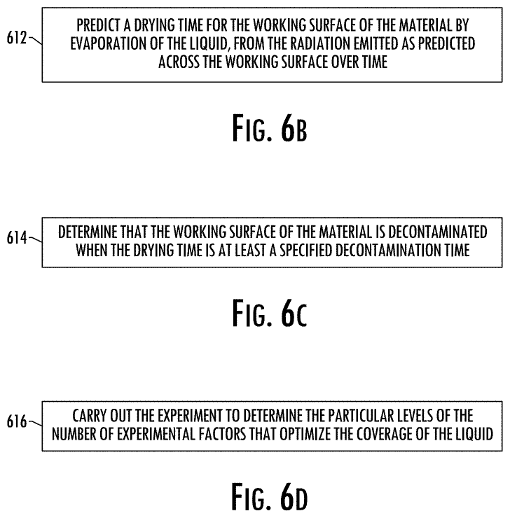

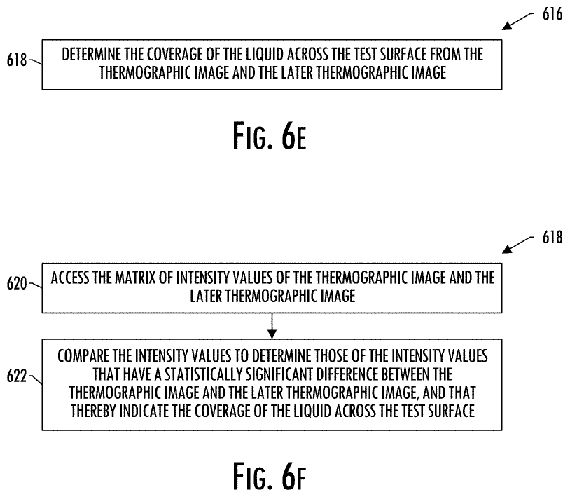

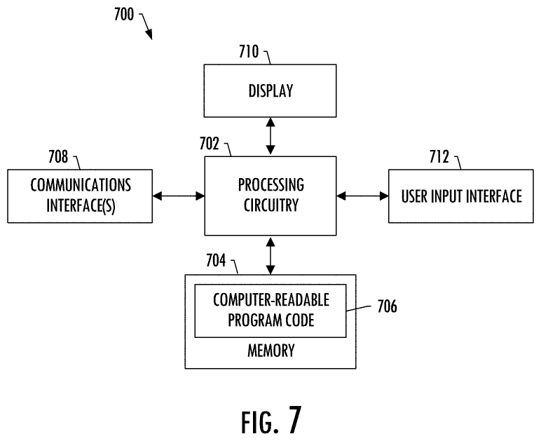

FIGS. 6A-6F are flowcharts illustrating various steps in a method 600 of decontaminating a surface of a material, according to various example implementations of the present disclosure. The method includes obtaining training data including a time-lapse of thermographic images of a test surface of the material on which a liquid that includes a decontamination agent is deposited, the thermographic images having a dot matrix data structure with a matrix of intensity values that describe radiation emitted and thereby indicate temperature across the test surface over time, as shown at block 602 of FIG. 6A. The method includes accessing a parametric model of the radiation emitted as a function of time, the parametric model including a fixed set of parameters with values that are unknown, as shown at block 604. The method includes applying a spatial regularization to the parametric model in which the fixed set of parameters are made functions of location across the test surface of the material, as shown at block 606. The method includes fitting the parametric model with the spatial regularization to the training data to estimate the values of the fixed set of parameters, and thereby produce a fitted parametric model, as shown at block 608. And the method includes deploying the fitted parametric model to predict the radiation emitted across a working surface of the material on which the liquid is deposited, the radiation emitted is predicted across the working surface over time, and decontamination of the working surface is determinable from the radiation emitted as predicted, as shown at block 610.

In some examples, the method 600 further includes predicting a drying time for the working surface of the material by evaporation of the liquid, from the radiation emitted as predicted across the working surface over time, as shown at block 612 of FIG. 6B.

In some examples, the method 600 further includes determining that the working surface of the material is decontaminated when the drying time is at least a specified decontamination time, as shown at block 614 of FIG. 6C.

In some examples, the training data is obtained at block 602 from an experiment designed to test an effect of a number of experimental factors on the radiation emitted across the test surface over time. In some of these examples, the fitted parametric model is deployed at block 610 to predict the radiation emitted under particular levels of the number of experimental factors.

In some examples, the experiment is also designed to test the effect of the number of experimental factors on coverage of the liquid across the test surface. In some of these examples, the method further includes carrying out the experiment to determine the particular levels of the number of experimental factors that optimize the coverage of the liquid, as shown at block 616 of FIG. 6D.

In some further examples, the thermographic images include a thermographic image captured before the liquid is deposited on the test surface, and a later thermographic image captured after the liquid is deposited on the test surface. And in some of these further examples, carrying out the experiment at block 616 includes determining the coverage of the liquid across the test surface from the thermographic image and the later thermographic image, as shown at block 618 of FIG. 6E.

In some examples, determining the coverage of the liquid across the test surface at block 618 includes accessing the matrix of intensity values of the thermographic image and the later thermographic image, as shown at block 620 of FIG. 6F. And in these examples, determining the coverage also includes comparing the intensity values to determine those of the intensity values that have a statistically significant difference between the thermographic image and the later thermographic image, and that thereby indicate the coverage of the liquid across the test surface, as shown at block 622.

In some examples, the liquid with the decontamination agent is deposited on the test surface in an environment and using an electrostatic sprayer. In some of these examples, the training data is obtained at block 602 from the experiment designed to test the effect of the number of experimental factors including multiple ones of the material, a profile of the test surface of the material, a temperature of the environment, a humidity of the environment, the electrostatic sprayer, the liquid, or an orientation of the electrostatic sprayer with respect to the test surface when the liquid with the decontamination agent is deposited.

Example implementations of the present disclosure may be implemented by various means. These means may include computer hardware, alone or under direction of one or more computer programs from a computer-readable storage medium. In some examples, one or more apparatuses such as one or more of the computers 216, 218, 220, 224, 226 and 230 may be configured to implement example implementations of the present disclosure. In examples involving more than one apparatus, the respective apparatuses may be connected to or otherwise in communication with one another in a number of different manners, such as directly or indirectly via a wired or wireless network or the like.

FIG. 7 illustrates an apparatus 700 according to some example implementations of the present disclosure. Generally, an apparatus of exemplary implementations of the present disclosure may comprise, include or be embodied in one or more fixed or portable electronic devices. Examples of suitable electronic devices include a smartphone, tablet computer, laptop computer, desktop computer, workstation computer, server computer or the like. The apparatus may include one or more of each of a number of components such as, for example, processing circuitry 702 (e.g., processor unit) connected to a memory 704 (e.g., storage device).

The processing circuitry 702 may be composed of one or more processors alone or in combination with one or more memories. The processing circuitry is generally any piece of computer hardware that is capable of processing information such as, for example, data, computer programs and/or other suitable electronic information. The processing circuitry is composed of a collection of electronic circuits some of which may be packaged as an integrated circuit or multiple interconnected integrated circuits (an integrated circuit at times more commonly referred to as a “chip”). The processing circuitry may be configured to execute computer programs, which may be stored onboard the processing circuitry or otherwise stored in the memory 704 (of the same or another apparatus).

The processing circuitry 702 may be a number of processors, a multi-core processor or some other type of processor, depending on the particular implementation. Further, the processing circuitry may be implemented using a number of heterogeneous processor systems in which a main processor is present with one or more secondary processors on a single chip. As another illustrative example, the processing circuitry may be a symmetric multi-processor system containing multiple processors of the same type. In yet another example, the processing circuitry may be embodied as or otherwise include one or more ASICs, FPGAs or the like. Thus, although the processing circuitry may be capable of executing a computer program to perform one or more functions, the processing circuitry of various examples may be capable of performing one or more functions without the aid of a computer program. In either instance, the processing circuitry may be appropriately programmed to perform functions or operations according to example implementations of the present disclosure.

The memory 704 is generally any piece of computer hardware that is capable of storing information such as, for example, data, computer programs (e.g., computer-readable program code 706) and/or other suitable information either on a temporary basis and/or a permanent basis. The memory may include volatile and/or non-volatile memory, and may be fixed or removable. Examples of suitable memory include random access memory (RAM), read-only memory (ROM), a hard drive, a flash memory, a thumb drive, a removable computer diskette, an optical disk, a magnetic tape or some combination of the above. Optical disks may include compact disk-read only memory (CD-ROM), compact disk-read/write (CD-R/W), DVD or the like. In various instances, the memory may be referred to as a computer-readable storage medium. The computer-readable storage medium is a non-transitory device capable of storing information, and is distinguishable from computer-readable transmission media such as electronic transitory signals capable of carrying information from one location to another. Computer-readable medium as described herein may generally refer to a computer-readable storage medium or computer-readable transmission medium.

In addition to the memory 704, the processing circuitry 702 may also be connected to one or more interfaces for displaying, transmitting and/or receiving information. The interfaces may include a communications interface 708 (e.g., communications unit) and/or one or more user interfaces. The communications interface may be configured to transmit and/or receive information, such as to and/or from other apparatus(es), network(s) or the like. The communications interface may be configured to transmit and/or receive information by physical (wired) and/or wireless communications links. Examples of suitable communication interfaces include a network interface controller (NIC), wireless NIC (WNIC) or the like.

The user interfaces may include a display 710 and/or one or more user input interfaces 712 (e.g., input/output unit). The display may be configured to present or otherwise display information to a user, suitable examples of which include a liquid crystal display (LCD), light-emitting diode display (LED), plasma display panel (PDP) or the like. The user input interfaces may be wired or wireless, and may be configured to receive information from a user into the apparatus, such as for processing, storage and/or display. Suitable examples of user input interfaces include a microphone, image or video capture device, keyboard or keypad, joystick, touch-sensitive surface (separate from or integrated into a touchscreen), biometric sensor or the like. The user interfaces may further include one or more interfaces for communicating with peripherals such as printers, scanners or the like.

As indicated above, program code instructions may be stored in memory, and executed by processing circuitry that is thereby programmed, to implement functions of the systems, subsystems, tools and their respective elements described herein. As will be appreciated, any suitable program code instructions may be loaded onto a computer or other programmable apparatus from a computer-readable storage medium to produce a particular machine, such that the particular machine becomes a means for implementing the functions specified herein. These program code instructions may also be stored in a computer-readable storage medium that can direct a computer, a processing circuitry or other programmable apparatus to function in a particular manner to thereby generate a particular machine or particular article of manufacture. The instructions stored in the computer-readable storage medium may produce an article of manufacture, where the article of manufacture becomes a means for implementing functions described herein. The program code instructions may be retrieved from a computer-readable storage medium and loaded into a computer, processing circuitry or other programmable apparatus to configure the computer, processing circuitry or other programmable apparatus to execute operations to be performed on or by the computer, processing circuitry or other programmable apparatus.

Retrieval, loading and execution of the program code instructions may be performed sequentially such that one instruction is retrieved, loaded and executed at a time. In some example implementations, retrieval, loading and/or execution may be performed in parallel such that multiple instructions are retrieved, loaded, and/or executed together. Execution of the program code instructions may produce a computer-implemented process such that the instructions executed by the computer, processing circuitry or other programmable apparatus provide operations for implementing functions described herein.

Execution of instructions by a processing circuitry, or storage of instructions in a computer-readable storage medium, supports combinations of operations for performing the specified functions. In this manner, an apparatus 700 may include a processing circuitry 702 and a computer-readable storage medium or memory 704 coupled to the processing circuitry, where the processing circuitry is configured to execute computer-readable program code 706 stored in the memory. It will also be understood that one or more functions, and combinations of functions, may be implemented by special purpose hardware-based computer systems and/or processing circuitry which perform the specified functions, or combinations of special purpose hardware and program code instructions.

As explained above and reiterated below, the present disclosure includes, without limitation, the following example implementations.

Clause 1. An apparatus for decontaminating a surface of a material, the apparatus comprising: a memory configured to store computer-readable program code; and processing circuitry configured to access the memory, and execute the computer-readable program code to cause the apparatus to at least: obtain training data including a time-lapse of thermographic images of a test surface of the material on which a liquid that includes a decontamination agent is deposited, the thermographic images having a dot matrix data structure with a matrix of intensity values that describe radiation emitted and thereby indicate temperature across the test surface over time; access a parametric model of the radiation emitted as a function of time, the parametric model including a fixed set of parameters with values that are unknown; apply a spatial regularization to the parametric model in which the fixed set of parameters are made functions of location across the test surface of the material, fit the parametric model with the spatial regularization to the training data to estimate the values of the fixed set of parameters, and thereby produce a fitted parametric model; and deploy the fitted parametric model to predict the radiation emitted across a working surface of the material on which the liquid is deposited, the radiation emitted is predicted across the working surface over time, and decontamination of the working surface is determinable from the radiation emitted as predicted.

Clause 2. The apparatus of clause 1, wherein the processing circuitry is configured to execute the computer-readable program code to cause the apparatus to further predict a drying time for the working surface of the material by evaporation of the liquid, from the radiation emitted as predicted across the working surface over time.

Clause 3. The apparatus of clause 2, wherein the processing circuitry is configured to execute the computer-readable program code to cause the apparatus to further determine that the working surface of the material is decontaminated when the drying time is at least a specified decontamination time.

Clause 4. The apparatus of any of clauses 1 to 3, wherein the training data is obtained from an experiment designed to test an effect of a number of experimental factors on the radiation emitted across the test surface over time, and the fitted parametric model is deployed to predict the radiation emitted under particular levels of the number of experimental factors.

Clause 5. The apparatus of clause 4, wherein the experiment is also designed to test the effect of the number of experimental factors on coverage of the liquid across the test surface, and the processing circuitry is configured to execute the computer-readable program code to cause the apparatus to further carry out the experiment to determine the particular levels of the number of experimental factors that optimize the coverage of the liquid.

Clause 6. The apparatus of clause 5, wherein the thermographic images include a thermographic image captured before the liquid is deposited on the test surface, and a later thermographic image captured after the liquid is deposited on the test surface, and wherein the apparatus caused to carry out the experiment includes the apparatus caused to determine the coverage of the liquid across the test surface from the thermographic image and the later thermographic image.

Clause 7. The apparatus of clause 6, wherein the apparatus caused to determine the coverage of the liquid across the test surface includes the apparatus caused to: access the matrix of intensity values of the thermographic image and the later thermographic image; and compare the intensity values to determine those of the intensity values that have a statistically significant difference between the thermographic image and the later thermographic image, and that thereby indicate the coverage of the liquid across the test surface.

Clause 8. The apparatus of any of clauses 4 to 7, wherein the liquid with the decontamination agent is deposited on the test surface in an environment and using an electrostatic sprayer, and wherein the training data is obtained from the experiment designed to test the effect of the number of experimental factors including multiple ones of the material, a profile of the test surface of the material, a temperature of the environment, a humidity of the environment, the electrostatic sprayer, the liquid, or an orientation of the electrostatic sprayer with respect to the test surface when the liquid with the decontamination agent is deposited.

Clause 9. A method of decontaminating a surface of a material, the method comprising: obtaining training data including a time-lapse of thermographic images of a test surface of the material on which a liquid that includes a decontamination agent is deposited, the thermographic images having a dot matrix data structure with a matrix of intensity values that describe radiation emitted and thereby indicate temperature across the test surface over time; accessing a parametric model of the radiation emitted as a function of time, the parametric model including a fixed set of parameters with values that are unknown; applying a spatial regularization to the parametric model in which the fixed set of parameters are made functions of location across the test surface of the material; fitting the parametric model with the spatial regularization to the training data to estimate the values of the fixed set of parameters, and thereby produce a fitted parametric model; and deploying the fitted parametric model to predict the radiation emitted across a working surface of the material on which the liquid is deposited, the radiation emitted is predicted across the working surface over time, and decontamination of the working surface is determinable from the radiation emitted as predicted.

Clause 10. The method of clause 9, wherein the method further comprises predicting a drying time for the working surface of the material by evaporation of the liquid, from the radiation emitted as predicted across the working surface over time.

Clause 11. The method of clause 10, wherein the method further comprises determining that the working surface of the material is decontaminated when the drying time is at least a specified decontamination time.

Clause 12. The method of any of clauses 9 to 11, wherein the training data is obtained from an experiment designed to test an effect of a number of experimental factors on the radiation emitted across the test surface over time, and the fitted parametric model is deployed to predict the radiation emitted under particular levels of the number of experimental factors.

Clause 13. The method of clause 12, wherein the experiment is also designed to test the effect of the number of experimental factors on coverage of the liquid across the test surface, and the method further comprises carrying out the experiment to determine the particular levels of the number of experimental factors that optimize the coverage of the liquid.

Clause 14. The method of clause 13, wherein the thermographic images include a thermographic image captured before the liquid is deposited on the test surface, and a later thermographic image captured after the liquid is deposited on the test surface, and wherein carrying out the experiment includes determining the coverage of the liquid across the test surface from the thermographic image and the later thermographic image.

Clause 15. The method of clause 14, wherein determining the coverage of the liquid across the test surface includes: accessing the matrix of intensity values of the thermographic image and the later thermographic image; and comparing the intensity values to determine those of the intensity values that have a statistically significant difference between the thermographic image and the later thermographic image, and that thereby indicate the coverage of the liquid across the test surface.

Clause 16. The method of any of clauses 12 to 15, wherein the liquid with the decontamination agent is deposited on the test surface in an environment and using an electrostatic sprayer, and wherein the training data is obtained from the experiment designed to test the effect of the number of experimental factors including multiple ones of the material, a profile of the test surface of the material, a temperature of the environment, a humidity of the environment, the electrostatic sprayer, the liquid, or an orientation of the electrostatic sprayer with respect to the test surface when the liquid with the decontamination agent is deposited.

Clause 17. A computer-readable storage medium for decontaminating a surface of a material, the computer-readable storage medium being non-transitory and having computer-readable program code stored therein that, in response to execution by processing circuitry, causes an apparatus to at least: obtain training data including a time-lapse of thermographic images of a test surface of the material on which a liquid that includes a decontamination agent is deposited, the thermographic images having a dot matrix data structure with a matrix of intensity values that describe radiation emitted and thereby indicate temperature across the test surface over time; access a parametric model of the radiation emitted as a function of time, the parametric model including a fixed set of parameters with values that are unknown; apply a spatial regularization to the parametric model in which the fixed set of parameters are made functions of location across the test surface of the material; fit the parametric model with the spatial regularization to the training data to estimate the values of the fixed set of parameters, and thereby produce a fitted parametric model, and deploy the fitted parametric model to predict the radiation emitted across a working surface of the material on which the liquid is deposited, the radiation emitted is predicted across the working surface over time, and decontamination of the working surface is determinable from the radiation emitted as predicted.

Clause 18. The computer-readable storage medium of clause 17, wherein the computer-readable storage medium has further computer-readable program code stored therein that, in response to execution by the processing circuitry, causes the apparatus to further predict a drying time for the working surface of the material by evaporation of the liquid, from the radiation emitted as predicted across the working surface over time.

Clause 19. The computer-readable storage medium of clause 18, wherein the computer-readable storage medium has further computer-readable program code stored therein that, in response to execution by the processing circuitry, causes the apparatus to further determine that the working surface of the material is decontaminated when the drying time is at least a specified decontamination time.

Clause 20. The computer-readable storage medium of any of clauses 17 to 19, wherein the training data is obtained from an experiment designed to test an effect of a number of experimental factors on the radiation emitted across the test surface over time, and the fitted parametric model is deployed to predict the radiation emitted under particular levels of the number of experimental factors.

Clause 21. The computer-readable storage medium of clause 20, wherein the experiment is also designed to test the effect of the number of experimental factors on coverage of the liquid across the test surface, and the computer-readable storage medium has further computer-readable program code stored therein that, in response to execution by the processing circuitry, causes the apparatus to further carry out the experiment to determine the particular levels of the number of experimental factors that optimize the coverage of the liquid.

Clause 22. The computer-readable storage medium of clause 21, wherein the thermographic images include a thermographic image captured before the liquid is deposited on the test surface, and a later thermographic image captured after the liquid is deposited on the test surface, and wherein the apparatus caused to carry out the experiment includes the apparatus caused to determine the coverage of the liquid across the test surface from the thermographic image and the later thermographic image.

Clause 23. The computer-readable storage medium of clause 22, wherein the apparatus caused to determine the coverage of the liquid across the test surface includes the apparatus caused to: access the matrix of intensity values of the thermographic image and the later thermographic image; and compare the intensity values to determine those of the intensity values that have a statistically significant difference between the thermographic image and the later thermographic image, and that thereby indicate the coverage of the liquid across the test surface.

Clause 24. The computer-readable storage medium of any of clauses 20 to 23, wherein the liquid with the decontamination agent is deposited on the test surface in an environment and using an electrostatic sprayer, and wherein the training data is obtained from the experiment designed to test the effect of the number of experimental factors including multiple ones of the material, a profile of the test surface of the material, a temperature of the environment, a humidity of the environment, the electrostatic sprayer, the liquid, or an orientation of the electrostatic sprayer with respect to the test surface when the liquid with the decontamination agent is deposited.

Many modifications and other implementations of the disclosure set forth herein will come to mind to one skilled in the art to which the disclosure pertains having the benefit of the teachings presented in the foregoing description and the associated figures. Therefore, it is to be understood that the disclosure is not to be limited to the specific implementations disclosed and that modifications and other implementations are intended to be included within the scope of the appended claims. Moreover, although the foregoing description and the associated figures describe example implementations in the context of certain example combinations of elements and/or functions, it should be appreciated that different combinations of elements and/or functions may be provided by alternative implementations without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

What is claimed is:

1. An apparatus for decontaminating a surface of a material, the apparatus comprising:
a memory configured to store computer-readable program code; and
processing circuitry configured to access the memory, and execute the computer-readable program code to cause the apparatus to at least:
obtain training data including a time-lapse of thermographic images of a test surface of the material on which a liquid that includes a decontamination agent is deposited, the thermographic images having a dot matrix data structure with a matrix of intensity values that describe radiation emitted and thereby indicate temperature across the test surface over time;
access a parametric model of the radiation emitted as a function of time, the parametric model including a fixed set of parameters with values that are unknown;
apply a spatial regularization to the parametric model in which the fixed set of parameters are made functions of location across the test surface of the material;
fit the parametric model with the spatial regularization to the training data to estimate the values of the fixed set of parameters, and thereby produce a fitted parametric model; and
deploy the fitted parametric model to predict the radiation emitted across a working surface of the material on which the liquid is deposited, the radiation emitted is predicted across the working surface over time, and decontamination of the working surface is determinable from the radiation emitted as predicted.

2. The apparatus of claim 1, wherein the processing circuitry is configured to execute the computer-readable program code to cause the apparatus to further predict a drying time for the working surface of the material by evaporation of the liquid, from the radiation emitted as predicted across the working surface over time.

3. The apparatus of claim 2, wherein the processing circuitry is configured to execute the computer-readable program code to cause the apparatus to further determine that the working surface of the material is decontaminated when the drying time is at least a specified decontamination time.

4. The apparatus of claim 1, wherein the training data is obtained from an experiment designed to test an effect of a number of experimental factors on the radiation emitted across the test surface over time, and the fitted parametric model is deployed to predict the radiation emitted under particular levels of the number of experimental factors.

5. The apparatus of claim 4, wherein the experiment is also designed to test the effect of the number of experimental factors on coverage of the liquid across the test surface, and the processing circuitry is configured to execute the computer-readable program code to cause the apparatus to further carry out the experiment to determine the particular levels of the number of experimental factors that optimize the coverage of the liquid.

6. The apparatus of claim 5, wherein the thermographic images include a thermographic image captured before the liquid is deposited on the test surface, and a later thermographic image captured after the liquid is deposited on the test surface, and
wherein the apparatus caused to carry out the experiment includes the apparatus caused to determine the coverage of the liquid across the test surface from the thermographic image and the later thermographic image.

7. The apparatus of claim 4, wherein the liquid with the decontamination agent is deposited on the test surface in an environment and using an electrostatic sprayer, and
wherein the training data is obtained from the experiment designed to test the effect of the number of experimental factors including multiple ones of the material, a profile of the test surface of the material, a temperature of the environment, a humidity of the environment, the electrostatic sprayer, the liquid, or an orientation of the electrostatic sprayer with respect to the test surface when the liquid with the decontamination agent is deposited.

8. A method of decontaminating a surface of a material, the method comprising:
obtaining training data including a time-lapse of thermographic images of a test surface of the material on which a liquid that includes a decontamination agent is deposited, the thermographic images having a dot matrix data structure with a matrix of intensity values that describe radiation emitted and thereby indicate temperature across the test surface over time;
accessing a parametric model of the radiation emitted as a function of time, the parametric model including a fixed set of parameters with values that are unknown;
applying a spatial regularization to the parametric model in which the fixed set of parameters are made functions of location across the test surface of the material;
fitting the parametric model with the spatial regularization to the training data to estimate the values of the fixed set of parameters, and thereby produce a fitted parametric model; and
deploying the fitted parametric model to predict the radiation emitted across a working surface of the material on which the liquid is deposited, the radiation emitted is predicted across the working surface over time, and decontamination of the working surface is determinable from the radiation emitted as predicted.

9. The method of claim 8, wherein the method further comprises predicting a drying time for the working surface of the material by evaporation of the liquid, from the radiation emitted as predicted across the working surface over time.

10. The method of claim 9, wherein the method further comprises determining that the working surface of the material is decontaminated when the drying time is at least a specified decontamination time.

11. The method of claim 8, wherein the training data is obtained from an experiment designed to test an effect of a number of experimental factors on the radiation emitted across the test surface over time, and the fitted parametric model is deployed to predict the radiation emitted under particular levels of the number of experimental factors.

12. The method of claim 11, wherein the experiment is also designed to test the effect of the number of experimental factors on coverage of the liquid across the test surface, and the method further comprises carrying out the experiment to determine the particular levels of the number of experimental factors that optimize the coverage of the liquid.

13. The method of claim 12, wherein the thermographic images include a thermographic image captured before the liquid is deposited on the test surface, and a later thermographic image captured after the liquid is deposited on the test surface, and
wherein carrying out the experiment includes determining the coverage of the liquid across the test surface from the thermographic image and the later thermographic image.

14. The method of claim 11, wherein the liquid with the decontamination agent is deposited on the test surface in an environment and using an electrostatic sprayer, and
wherein the training data is obtained from the experiment designed to test the effect of the number of experimental factors including multiple ones of the material, a profile of the test surface of the material, a temperature of the environment, a humidity of the environment, the electrostatic sprayer, the liquid, or an orientation of the electrostatic sprayer with respect to the test surface when the liquid with the decontamination agent is deposited.

15. A computer-readable storage medium for decontaminating a surface of a material, the computer-readable storage medium being non-transitory and having computer-readable program code stored therein that, in response to execution by processing circuitry, causes an apparatus to at least:
obtain training data including a time-lapse of thermographic images of a test surface of the material on which a liquid that includes a decontamination agent is deposited, the thermographic images having a dot matrix data structure with a matrix of intensity values that describe radiation emitted and thereby indicate temperature across the test surface over time;
access a parametric model of the radiation emitted as a function of time, the parametric model including a fixed set of parameters with values that are unknown;
apply a spatial regularization to the parametric model in which the fixed set of parameters are made functions of location across the test surface of the material;
fit the parametric model with the spatial regularization to the training data to estimate the values of the fixed set of parameters, and thereby produce a fitted parametric model; and
deploy the fitted parametric model to predict the radiation emitted across a working surface of the material on which the liquid is deposited, the radiation emitted is predicted across the working surface over time, and decontamination of the working surface is determinable from the radiation emitted as predicted.

16. The computer-readable storage medium of claim 15, wherein the computer-readable storage medium has further computer-readable program code stored therein that, in response to execution by the processing circuitry, causes the apparatus to further predict a drying time for the working surface of the material by evaporation of the liquid, from the radiation emitted as predicted across the working surface over time.

17. The computer-readable storage medium of claim 16, wherein the computer-readable storage medium has further computer-readable program code stored therein that, in response to execution by the processing circuitry, causes the apparatus to further determine that the working surface of the material is decontaminated when the drying time is at least a specified decontamination time.

18. The computer-readable storage medium of claim 15, wherein the training data is obtained from an experiment designed to test an effect of a number of experimental factors on the radiation emitted across the test surface over time, and the fitted parametric model is deployed to predict the radiation emitted under particular levels of the number of experimental factors.

19. The computer-readable storage medium of claim 18, wherein the experiment is also designed to test the effect of the number of experimental factors on coverage of the liquid across the test surface, and the computer-readable storage medium has further computer-readable program code stored therein that, in response to execution by the processing circuitry, causes the apparatus to further carry out the experiment to determine the particular levels of the number of experimental factors that optimize the coverage of the liquid.

20. The computer-readable storage medium of claim 19, wherein the thermographic images include a thermographic image captured before the liquid is deposited on the test surface, and a later thermographic image captured after the liquid is deposited on the test surface, and
wherein the apparatus caused to carry out the experiment includes the apparatus caused to determine the coverage of the liquid across the test surface from the thermographic image and the later thermographic image.
